(19) United States
(12) Patent Application Publication
(10) Pub. No.: US 2008/0262383 A1
(43) Pub. Date: Oct. 23, 2008

(54) NEEDLE ASSEMBLY WITH SEPARABLE HANDLE

(75) Inventors: Ronald Routhier, Attleboro, MA (US); Richard Tripp, Foxborough, MA (US); C. Russell Small, Rehoboth, MA (US); Joseph Antocci, Leominster, MA (US)

Correspondence Address: JOHN J. BROOKS, III, 20 WYSTERIA WAY, WRENTHAM, MA, 02093, US

(73) Assignee: NEEDLETECH PRODUCTS, INC., Attleboro, MA (US)

(21) Appl. No.: 12/101,128

(22) Filed: Apr. 10, 2008

Related U.S. Application Data
(60) Provisional application No. 60/912,422, filed on Apr. 17, 2007.

(51) Int. Cl. A61B 10/02

(52) U.S. Cl. 600/567

(57) ABSTRACT

A needle assembly comprising an outer cannula having a restraining element, an inner rod capable of being removably inserted into a hollow cross-section of the outer cannula, a handle having a flexible restraining member with a restraining nub and a protruding element whereby a force applied to the protruding element disengages the restraining nub from the outer cannula restraining element and the handle can be removed and attached to the outer cannula. Other embodiments of the invention comprise a strike cap integrally connected to the inner rod whereby a force applied to the strike cap is directly transferred to the inner rod. Other embodiments of the invention comprise a rod attachment member connected to the inner rod and the handle having at least one handle attachment element whereby the inner rod attachment member cooperates with the handle attachment element to removably attach the inner rod to the handle.

### CROSS-REFERENCE TO RELATED APPLICATIONS

This application claims the benefit of the following provisional application, which is hereby incorporated by reference in its entirety: Ser. No. 60/912,422 NEEDLE ASSEMBLY WITH SEPERABLE HANDLE, filed 17 Apr., 2007.

### STATEMENT REGARDING FEDERALLY SPONSORED RESEARCH OR DEVELOPMENT:

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

The present invention relates in general to multi-part needles and, more particularly, to a needle device with a removable handle.

2. Background Art

Needle assemblies have been known in the art for many years. One type of needle assembly, the biopsy device, has been historically used to obtain tissue samples from a living being. Theses biopsy devices typically include a hollow outer cannula with some form of an inner rod slidable within the outer cannula. The outer cannula conventionally consists of a proximal end, a distal end, and some form of a handle associated with the proximal end. The inner rod may typically take several different forms, including a sharpened stylet for insertion of the biopsy device into a patient, an inner cannula for sampling tissue, and/or an ejector rod for forcing the sample out of the inner cannula. The inner rod also typically includes a second or connection handle which may be secured to the handle of the outer cannula.

To obtain a tissue sample, the distal ends of the cannula and a sharpened inner rod, or stylet, are inserted through the patient's body and placed in a location from which to retrieve the tissue sample. The cannula and inner rod are then advanced into the tissue region from which a sample is desired. For soft tissue this advancement can be done easily. For samples that are beneath hard tissue, such as bone marrow that is accessible only through the bone, this advancement is accomplished with considerable force. After the distal ends of the cannula and inner rod are placed at the desired location, the sharpened rod may be replaced by a device that is capable of sampling tissue. Once the tissue sample is gathered, the entire assembly is removed from the patient.

Historically, this sampling has been performed as a single operation. However, modern applications of these needle assemblies have expanded to include using such devices to repair damage bone such as osteoporotic bone fractures or crushed vertebra. In this type of operation, typically, the cannula and rod portions of the biopsy device are inserted into the selected vertebra. After the cannula has been positioned within a vertebra, then an imaging device, such as an x-ray machine, is used to view the location of the cannula tip to determine whether it is positioned properly. If the cannula is not in the desired position, then the position of the biopsy device is adjusted as needed. Once it is determined that the cannula is positioned properly, then the inner rod is removed, and a form of cement or other binding element is injected into the vertebra through the cannula in order to reinforce the crushed vertebra. After the cement is injected, the needle assembly needs to be safely removed from the patient.

In these modern applications, it is also possible that the assembly can be used in close proximity to other assemblies or other devices.

Some changes have been made to devices in the art to respond to these new uses. Some of the changes involve the ability to remove handles from elements such as the cannula. Some of these embodiments with removable handles require rotation of one of the handles themselves to unlock and separate the handles from one another. Other embodiments of removable handles are implemented with a locknut securing device positioned directly atop the inner rod allowing the user to directly remove the rod itself from the interior of the cannula.

None of the above embodiments, taken either singularly or in combination, is seen to describe embodiments of the present invention.

### BRIEF SUMMARY OF THE INVENTION

The present invention has multiple aspects, formats, and applications. One embodiment of the invention provides a needle assembly comprising an outer cannula having a restraining element, a handle, a restraining member operably attached to the handle and the restraining member having a restraining nub whereby the restraining nub engages with the restraining element and the handle can be removably attached to the outer cannula.

In another embodiment of the invention, the restraining member further comprises a flexible element having a protruding element whereby a force applied to the protruding element disengages the restraining nub from the restraining element and the handle can be removably attached to the outer cannula.

In still another embodiment of the invention, the handle can be held by at least one appendage of a hand while the force is applied to the protruding element by at least one appendage of the hand.

In a further embodiment of the invention, the outer diameter of the outer cannula insert is rounded whereby it can be manufactured by a turning means.

In another embodiment of the invention, the outer cannula insert is manufactured from metal.

In still another embodiment of the invention, the outer cannula comprises a distal end and a threaded proximal end.

In a further embodiment of the invention, the outer cannula comprises a distal end and a proximal end shaped as a luer fitting.

In another embodiment of the invention, the needle assembly further comprises a substantially hollow cross-section between a distal end and a proximal end of the outer cannula and an inner rod capable of being inserted into and retracted from the hollow cross-section of the outer cannula

In still another embodiment of the invention, the inner rod further comprises an inner rod proximal end and an inner rod distal end and a rigid rod between the ends and a strike cap integrally connected to the rigid rod whereby a force applied to the strike cap is directly transferred to the rigid rod.

In a further embodiment of the invention, the needle assembly further comprises a rod attachment member connected to the inner rod proximal end, the rod attachment member having at least one inner rod attachment element and the handle having at least one handle attachment element whereby the inner rod attachment element cooperates with the handle attachment element to removably attached the inner rod to the handle.

In still another embodiment of the invention, the rod attachment member is non-circular shaped whereby it provides a gripping surface for removable attachment of the inner rod to the handle.

In another embodiment of the invention, the needle assembly comprises an inner rod having an inner rod proximal end and an inner rod distal end, a rigid rod between the inner rod proximal end and inner rod distal end and a strike cap integrally connected to the rigid rod whereby a force applied to the strike cap is directly transferred to the rigid rod.

In still another embodiment of the invention, the needle assembly further comprises an outer cannula having a proximal end, a distal end and a substantially hollow cross-section between the proximal and distal ends, the substantially hollow cross-section being capable of removably receiving the rigid rod, a handle, a rod attachment member connected to the inner rod proximal end, the rod attachment member having at least one inner rod attachment element and the handle having at least one handle attachment element whereby the inner rod attachment element cooperates with the handle attachment element to removably attached the inner rod to the handle.

In still another embodiment of the invention, the needle assembly further comprises the outer cannula having a restraining element, a restraining member operably attached to the handle and the restraining member having a restraining nub whereby the restraining nub engages with the restraining element and the handle can be removably attached to the outer cannula.

In a further embodiment of the invention, the restraining member further comprises a flexible element with an attachment end connected to the handle and a non-attached end having a protruding element whereby a force applied to the protruding element disengages the restraining nub from the restraining element and the handle can be removably attached to the outer cannula.

### BRIEF DESCRIPTION OF THE SEVERAL VIEWS OF THE DRAWINGS

FIG. 1 shows a side perspective view of one embodiment of the needle assembly;

FIGS. 2A and 2B show a side perspective view of two alternative embodiments, not necessarily of a similar scale, of the outer cannula;

FIG. 3 shows side perspective view of one embodiment of the inner rod;

FIGS. 4A and 4B show two side perspective views of one embodiment of the handle;

FIG. 5 shows a perspective view of one embodiment of the restraining member;

FIG. 6 shows a side perspective view of an alternative embodiment of the restraining member and handle; and

FIG. 7 shows a side perspective view of one embodiment of the handle and restraining member creating a handle assembly.

### DETAILED DESCRIPTION OF THE INVENTION

While this invention is capable of being implemented in many different embodiments, there is shown in the drawings, and will herein be described in detail, several specific embodiments, with the understanding that the present disclosure can be considered as an exemplification of the principals of the invention and is not intended to limit the invention to the embodiments illustrated.

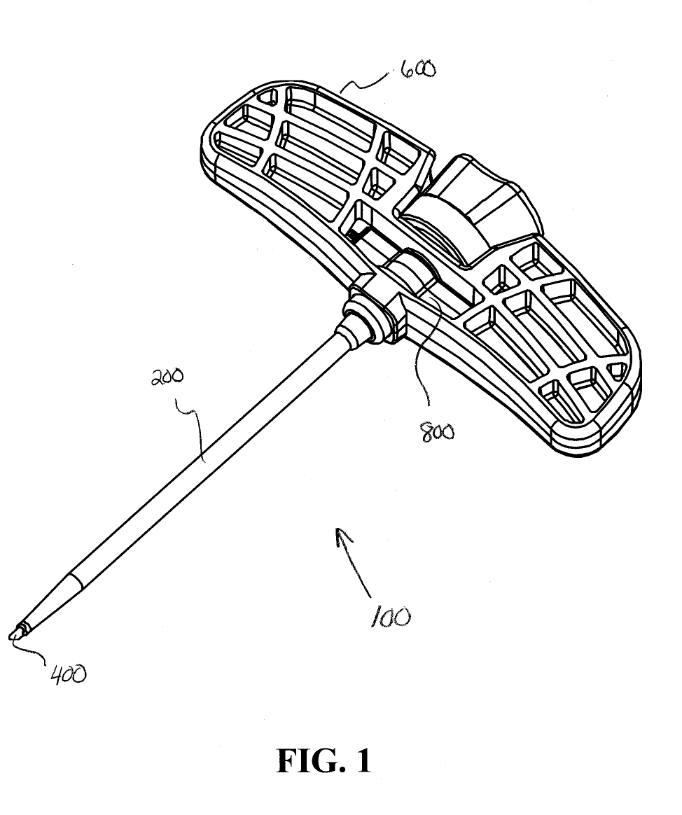

One embodiment of the needle assembly 100 is shown in FIG. 1 as comprising an outer cannula 200, an inner rod 400, a handle 600, and a restraining member 800. Embodiments of these elements are described in more detail below.

The Outer Cannula:

Generally, the outer cannula 200 is used in this assembly as a conduit to receive and pass tools and material through the needle assembly 100.

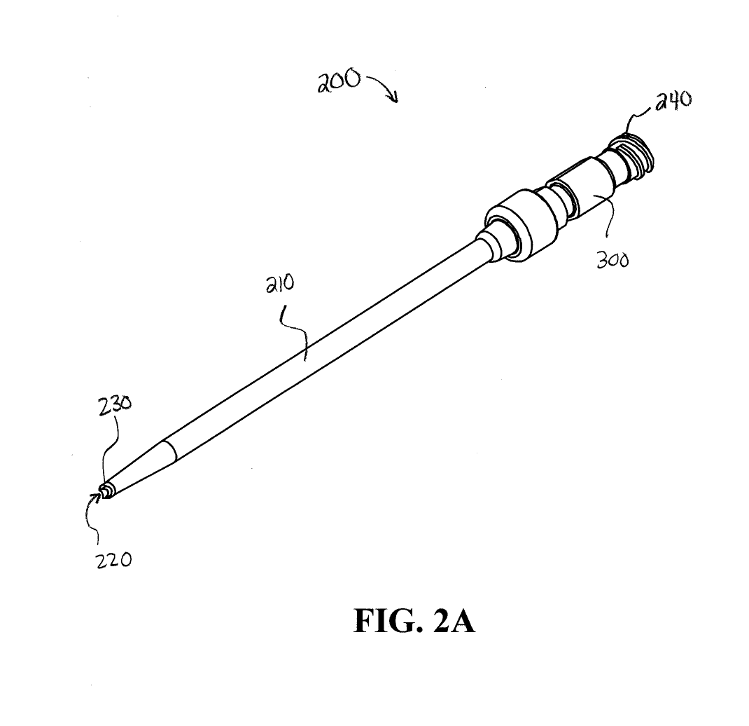

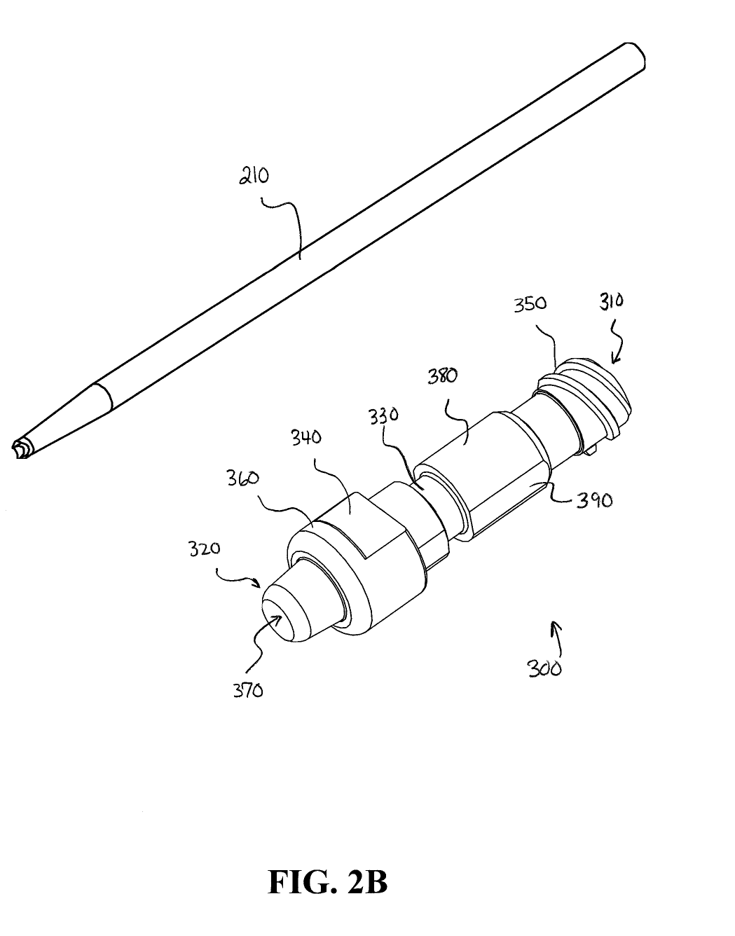

As shown in FIG. 2A, one embodiment of the outer cannula 200 is a hollow cylindrical element 210 with an interior bore 220 that extends from an outer cannula distal end 230 to an outer cannula proximal end 240. The interior bore 220 is somewhat larger in diameter than, and is not as long as, a rigid cylindrical rod 430 which is described later in this description. The size of the interior bore 220 permits a user, such as a physician, to insert, slide and rotate the outer cannula 200 relative to elements such as the inner rod 400 and vice versa. Desirably, but not necessarily, the outer cannula 200 is made of metal and contains markings along its outer surface to indicate the depth at which it is placed into a tissue such as a human or animal patient's body tissue. The outer cannula 200 can be various other lengths, depending upon the thickness of the tissue where it will be used.

The outer cannula distal end 230 is rigid and shaped to be easily inserted into a tissue. Usual, but not all embodiments of the outer cannula distal end 230 include a sharpening of the edges of the outer cannula's distal end 230.

The outer cannula proximal end 240 further comprises an outer cannula insert 300 capable of fitting securely with the handle 600. The outer cannula insert 300 may be a component integral to the outer cannula 200, or the outer cannula insert may be a separate element that is attached to the hollow cylindrical element 210 of the outer cannula 200. FIGS. 2A and 2B show different embodiments of the outer cannula 200 and outer cannula insert 300. The outer cannula insert 300 shown in FIG. 2A is integral with the outer cannula 200. FIG. 2B illustrates an embodiment of the invention wherein the cylindrical element 210 is an element separate from the outer cannula insert 300 until they are attached to create the outer cannula 200. These different embodiments provide flexibility for the manufacture of the outer cannula 200 and allow for the cylindrical element 210 and the outer cannula insert 300 to have different properties if desired. The attachment or integral manufacture of the outer cannula insert 300 with the cylindrical element 210 should be rigid and minimize movement of the outer cannula insert 300 in relation to the cylindrical element 210 when used operationally and must allow the receipt of the inner rod 400 through a means such as a insert hole or insert bore 370 cooperating with the interior bore 220 of the outer cannula.

As illustrated in FIG. 2B, one embodiment of the outer cannula insert 300 further comprises a cap receiving end 310, an outer cannula insert distal end 320, an outer cannula insert distal ring 360, an outer cannula insert body 380, one or more restraining elements 330 and one or more stabilizing elements 340.

The cap receiving end 310 of the outer cannula insert allows the outer cannula insert 300 to receive the inner rod 400. The cap receiving end 310 further comprises the insert bore 370 that extends the length of the outer cannula insert 300. The insert bore 370 operationally aligns with the hollow cylindrical element 210 and the interior bore 220 of the outer cannula 200.

The outer cannula insert distal end 320 is integral to, or rigidly attached to, the hollow cylindrical element 210 of the outer cannula 200. The outer diameter of a portion of the outer cannula insert 300 towards the outer cannula distal end 320 creates an outer cannula insert distal ring 360 which defines a diameter portion of the outer cannula insert 300 larger than the outer diameter of the remaining portion of the outer cannula insert 300. This larger diameter is shaped to engage the handle 600 and prevent complete insertion of the outer cannula insert 300 into the handle 600. The outer cannula insert distal ring 360 further comprises a stabilizing element 340. The stabilizing element 340 is a notched portion of the outer cannula insert 300. In the embodiment shown, the stabilizing element 340 is a notch out of distal ring 360 creating a ‘D’ shaped circumferential profile for a portion of the distal ring 360. This ‘D’ shaped stabilizing element 340 is capable of being inserted into the handle 600 and engages a complementary shaped stabilizing recess 650 in the handle 600. The engagement of the stabilizing element 340 with the handle 600 prevents axial movement and rotation of the outer cannula insert 300 relative to handle 600. The engagement of the stabilizing element 340 and the engagement of the distal ring 360 with the handle 600 assists in retaining the outer cannula insert 300 and the outer cannula 200 within the handle 600. Alternative embodiments of the outer cannula insert distal ring 360 include, but are not limited to other methods of restricting movement of the outer cannula insert within the handle 600 such as clips, pins, screws, nubs, other shapes or other protrusions. Alternative embodiments of the stabilizing element 340 include, but are not limited to other indentations, protrusions or other shapes which mate with a corresponding stabilizing recess 650 in the handle recess 640 of the handle 600.

The outer cannula insert 300 further comprises a outer cannula insert body 380 that comprises a closely toleranced diameter portion of the outer cannula insert 300 capable of being received into a correspondingly closely toleranced handle recess 640 in the handle 600 resulting in a minimal gap between the two when the body is inserted into the recess. The receipt of the outer cannula insert body 380 into the handle recess 640 provides additional stabilization of the outer cannula insert 300 within the handle 600. Indentations 390, as shown in FIG. 2B, on the outer cannula insert body are present in this embodiment where the outer cannula insert body is made by molds and the indentations are desirably, but not necessarily, designed along the mold seams to prevent the seams from interfering with the closely toleranced fit between the outer cannula insert body 380 and the handle recess 640. Alternative embodiments of the outer cannula insert body 380 include, but are not limited to other dimensional and outer profiles to include squares, rings, non-symmetrical, oblong and oval or other shapes that cooperate with a complementary recess.

The restraining element 330 engages the restraining member 800 restraining the outer cannula insert 300 and the outer cannula 200 relative to the handle 600. In this embodiment, the restraining element 330 comprises a reduced cylindrical diameter portion about the circumference of the outer cannula insert 300. This restraining element 330 is received within the handle 600 and provides a recess relative to the remaining body of the outer cannula insert 300, particularly relative to the outer cannula insert body 380, allowing the restraining element 330 to be engaged by the restraining member 800 described later in this description. Alternative embodiments of the restraining element 330 include other notches, recesses, protrusions or other shapes that can mate with a mating restraining member 800 to restrain the outer cannula insert 300 relative to the handle 600.

As shown in FIG. 2B, the outer cannula insert 300 may further comprise an outer cannula insert attachment means to receive a mating attachment member from an additional element or tool. In one embodiment, this attachment means comprises an outer cannula insert attachment element 350 comprising exposed threads to be engaged by matching female threads on an attachment member. It is also a contemplated embodiment of this invention for the attachment means to comprise luer lock type conical fittings in configurations per International Standard (ISO) 594/1 “Conical fittings with a 6% (Luer) taper for syringes, needles and certain other medical equipment—Part 1: General Requirements,” First Edition dated 1986-06-15 and International Standard (ISO) 594-2 “Conical fittings with a 6% (Luer) taper for syringes, needles and certain other medical equipment—Part 2: Lock Fittings,” Second Edition dated 1998-09-01, both Standards which are incorporated herein in their entirety, to be engaged by matching luer lock fittings on an attachment member from an additional element or tool. In embodiments consistent with ISO 594-2, the attachment means or outer cannula insert attachment element 350 can comprise either the male or female luer lock fittings comprised of lugs or threads. Embodiments of attachment means utilizing slip fittings, such as luer fittings described in ISO 594/1, are also contemplated. Other embodiments of the outer cannula insert attachment element 350 can include, but should not be limited to, attachment means such as clips, pins, screws, nubs or other protrusions that can cooperate with a mating attachment to removably connect an additional element or tool to the attachment element 350.

The outer cannula insert 300 and hollow cylindrical element 210 in the embodiments shown in FIG. 2A and FIG. 2B are desirably, but not necessarily, manufactured out of metal in a cylindrical shape. This shape and material is beneficial from a manufacturing point of view in that the outer cannula insert 300 can be manufactured using standard metal turning and milling processes and then be attached to the hollow cylindrical element 210. This shape and material composition also creates a beneficial embodiment because it provides a rigid material that can withstand the force applied to the needle assembly 100 while it is being inserted into the patient's body tissue. Other shapes, material composition and methods of manufacture of integral outer cannula inserts are possible.

In alternative embodiments, the outer cannula insert 300 can be manufactured from, but not limited to, other rigid materials such as plastics, epoxies, composites or resins. Other shapes, material composition and methods of manufacture of separate outer cannula inserts 300 are possible.

The Inner Rod:

Generally, the inner rod 400 serves as a rigid tool to help position the assembly 100 in the body of the patient. As is described below, embodiments of the assembly 100 also include using the inner rod 400 as a tool for extracting material from the body.

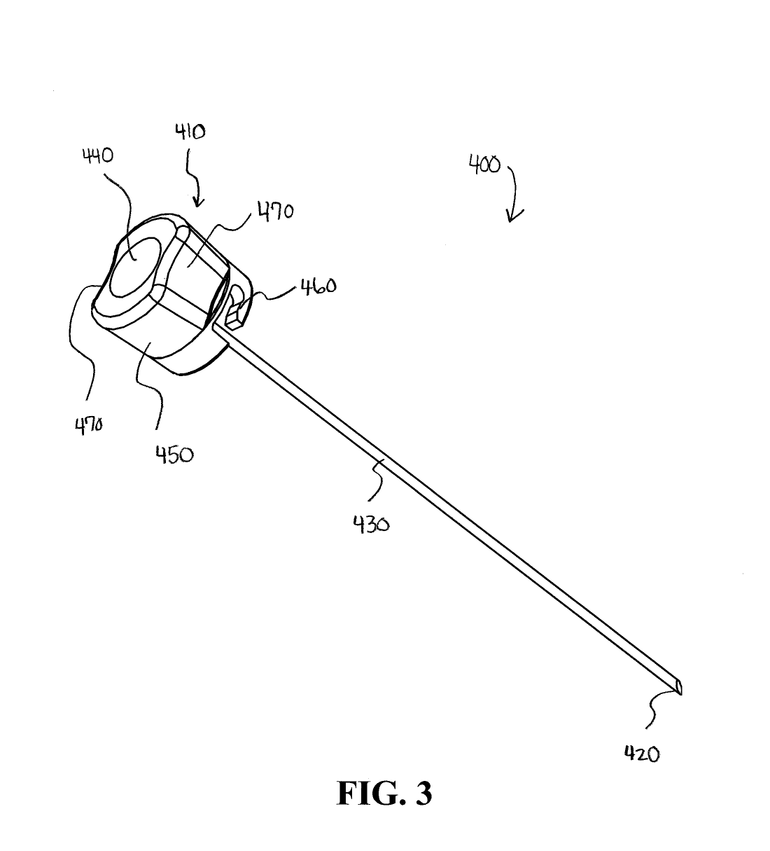

As shown in FIG. 3, one embodiment of the inner rod 400 comprises a rigid cylindrical rod 430 having an inner rod proximal end 410 and an inner rod distal end 420. The inner rod further comprises a strike cap 440 a rod attachment member 450 with one or more inner rod attachment elements 460.

The cylindrical rod 430 is shaped to be received within the interior bore 220 of the outer cannula 200 and extends from the inner rod distal end 420 to the inner rod proximal end 410. The inner rod distal end 420 of the cylindrical rod 430 is rigid and shaped to be inserted into tissue. Usual, but not all embodiments comprise the inner rod 400 being longer than outer cannula 200 with a sharpened inner rod distal end 420 which protrudes from outer cannula 200 to ease the insertion of the needle assembly 100 through the patient's body tissue. Additionally, the inner rod 400 may also be designed to assist in the extraction of material from the tissue. Examples of these alternative inner rod embodiments include, but are not limited to having a hollow inner cannula for sampling tissue, an ejector rod for forcing tissue from outer cannula, as well as other types of inner rods commonly used with such biopsy devices known by those of ordinary skill in the art. Desirably, the cylindrical rod 430 is comprised of a rigid material such as, but not necessarily metal. Other materials may be used for the cylindrical rod 430 that are capable of withstanding the force of the inner rod being struck while the needle assembly is being inserted into the patient's body tissue. Other rigid materials that could be used for the cylindrical rod 430 include, but are not limited to metals, plastics, composites or resins. Although the rigid cylindrical rod 430 as illustrated and described is cylindrical, it is also contemplated that the rigid cylindrical rod 403 can comprise other shapes of rigid rods, such as those having cross-sections about their diameter of, but not limited to square, rectangular, oblong, oval or a combination of those shapes.

The inner rod proximal end 410 provides means to attach the inner rod 400 to the handle 600 as well as provide a means to transfer force to the inner rod distal end 420. The inner rod proximal end 410 comprises a strike cap 440 and a rod attachment member 450 with an inner rod attachment element 460.

The strike cap 440 is a rigid element connected directly to the cylindrical rod 430 wherein the inner rod 400 is able to be struck in operation and the force from the strike cap 440 is transferred directly to the cylindrical rod 430 down to the inner rod distal end 420. The strike cap 440 may, but need not be integral to the rod attachment member 450. Desirably, the strike cap 440 is made from rigid material such as metal, however, the strike cap 440 may be made from other rigid materials capable of withstanding the impact of a very hard object such as a hammer. Other rigid materials that could be used for the strike cap 440 include, but are not limited to metals, plastics, composites or resins.

The rod attachment member 450 is capable of attaching the inner rod 400 to the handle 600. The rod attachment member 450 is shaped so that the inner rod 400 and outer cannula 200 may be attached to the handle 600 simultaneously. In one embodiment, the rod attachment member 450 comprises a rounded knob with an inner rod attachment element 460 comprising a channel or circumferential recess to receive one or more handle attachment elements 670, such as raised protrusions, on the handle 600. When the inner rod 400 is inserted into the outer cannula insert 300, the rod attachment member 450 is aligned so that the inner rod attachment element 460 aligns with the handle attachment element 670. The rod attachment member 450 is then moved so that the handle attachment element 670 engages and secures the inner rod attachment element 460 attaching the inner rod 400 to the handle 600. Other embodiments of the rod attachment member 450 engaging the handle 600 are possible to include, but not to be limited to connections such as are possible with clips, threaded members, screws, bolts and other connection means.

Although the embodiment illustrated in FIG. 3 provides a means to attach the inner rod 400 to the handle 600, other embodiments are possible such as, but not limited to embodiments where the inner rod 400 is attached to the outer cannula insert 300 or other elements.

In one embodiment, the rod attachment member 450 further comprises a shaped rod attachment member 450 wherein it may be more easily gripped and manipulated. One embodiment is shown in FIG. 3 where the knob is rounded and includes one or more indentations 470 to provide a gripping surface that allows the user to more easily manipulate the rod attachment member 450. Other shapes of the rod attachment member 450 are possible to include gripping surfaces formed from rectangular shapes, shapes with frictionally engaged surfaces or shapes that can be engaged by a tool such as a wrench, a screwdriver or a key.

The Handle:

Generally, the handle 600 is used to help the user manipulate the assembly 100.

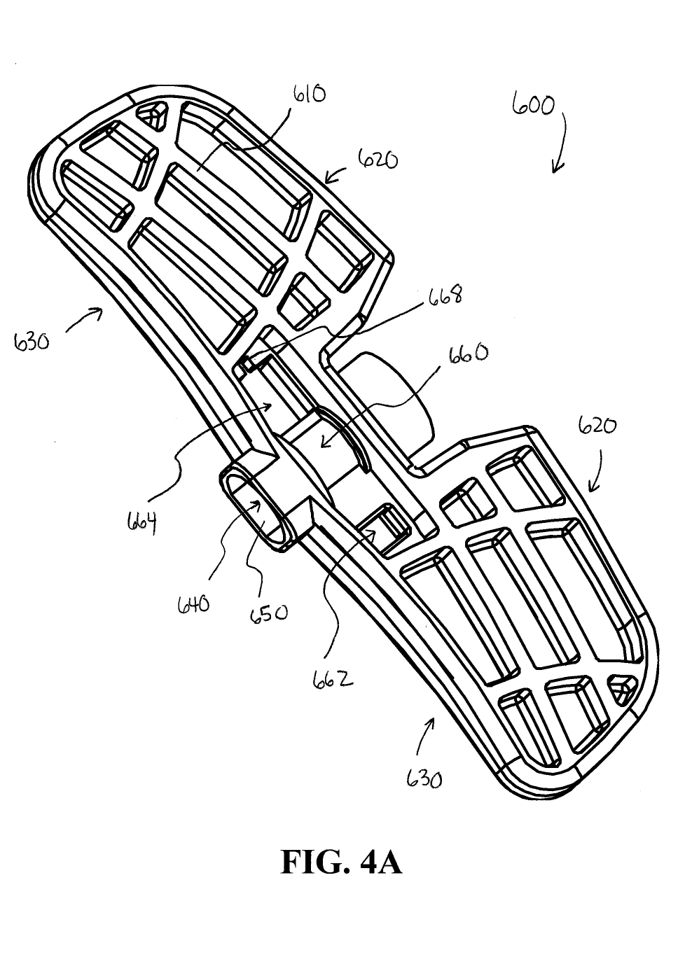

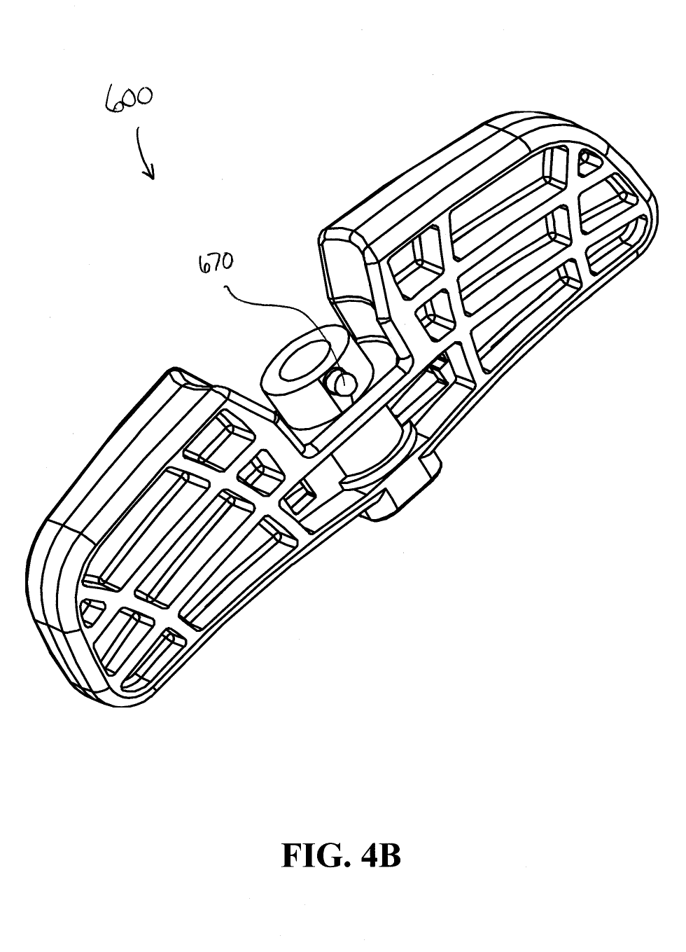

As shown in FIG. 4A and FIG. 4B, one embodiment of the handle 600 assists in the manipulation and insertion of the smaller elements of the needle assembly 100 and comprises a handle body 610, a handle recess 640 with a recess bottom portion 642, one or more stabilizing recesses 650, one or more handle attachment elements 670, an opening portion 660, a restraining member attachment hole 662 and a protruding element hole 664. As shown in FIG. 4A, in one embodiment, the handle body 610 is shaped to be used by a human hand comprising a smooth upper side 620 that can fit into the palm of a human's hand and a lower side 630 capable of being gripped by the fingers of the human hand. Although in this embodiment the handle 600 is shaped to fit the contours of a human hand, the handle 600 may also be shaped to fit the contours of other means of manipulating and inserting the needle assembly 100. These alternative embodiments of the handle 600 may be shaped to fit the contours of a robotic arm, a mechanical manipulator or other tools that assist in the manipulation and insertion of the needle assembly 100. Desirably, but not necessarily, the handle 600 is made from a molded or cast rigid plastic or metal material. Other rigid materials that could be used for the handle 600 include, but are not limited to composites, glass or resins.

The handle 600 also contains a handle recess 640 or bore to receive the outer cannula insert 300 and the inner rod 400. In one embodiment, the outer cannula insert 300 of the outer cannula 200 and the inner rod attachment member 450 mate with the handle through the handle recess 640. In this embodiment, the handle recess 640 is generally in the middle of the handle body 610 and the outer cannula insert 300 of the outer cannula 200 is received in the handle recess 640 through the recess bottom portion 642 of the handle recess 640. The handle recess 640 extends through the handle body 610 and allows the inner rod 400 to be inserted through the insert bore 370 of the outer cannula insert 300 and the interior bore 220 of the outer cannula 200.

The handle 600 further comprises a restraining member attachment hole 662 and a protruding element hole 664. The restraining member attachment hole 662 provides a means to attach the restraining member 800 to the handle 600. The protruding element hole 664 provides a means to allow the user of the needle assembly 100 to access and provide a force to a non-attached end 820 of the restraining member 800.

As shown in FIG. 4A, the handle 600 further comprises an opening portion 660 of the handle 600 that exposes a portion of the handle recess 640, making the restraining element 330 of the outer cannula insert 300 accessible when the outer cannula insert 300 is received into the handle recess 640.

The handle recess 640 further comprises a recess bottom portion 642 which has an inner diameter created that is of sufficient size to allow the insertion of the outer cannula insert cap receiving end 310, the outer cannula insert body 380 and the portion of the distal ring 360 that defines the outer cannula stabilizing element 340. The recess bottom portion 642 is also of a diameter small enough to prevent the insertion of the entire outer cannula insert distal ring 360. The recess bottom portion 642 receives the portion of the distal ring 360 that contains the stabilizing element 340 but prevents insertion of the outer cannula insert 300 beyond the stabilizing element 340. The inner diameter of the recess bottom portion 642 is also of a diameter larger than the other portions of the handle recess 640 which, when engaged with the larger diameter of the whole distal ring 360, further prevents insertion of the outer cannula insert 300 into the handle recess 640. Alternative embodiments of the recess bottom portion 642 include, but are not limited to other methods of restricting movement of the outer cannula insert 300 within the handle 600 through engagement with clips, pins, screws, nubs, other shapes or other protrusions on the outer cannula insert distal ring 360 or the outer cannula insert 300.

The handle 600 further comprises one or more stabilizing recesses 650 which mate with the stabilizing element 340 of the distal ring 360 of the outer cannula insert 300. In this embodiment, the stabilizing recess 650 comprises the handle recess bottom portion 642 having a ‘D’ shaped profile to receive the ‘D’ shaped profile of the stabilizing element 340 of the distal ring 360 on the outer cannula insert 300. The receipt of the stabilizing element 340 into the stabilizing recess 650 secures the outer cannula insert 300 and the outer cannula 200 to the handle 600 about the longitudinal axis of the outer cannula interior bore 220. This stabilization allows the handle 600 and the outer cannula 200 to be rigidly manipulated about the outer cannula bore axis while still allowing the handle 600 to be removed from the outer cannula insert 300 and the outer cannula 200. Other embodiments of the stabilizing recess 650 include but are not limited to shapes that will securely receive the profile of the distal ring 360 on the outer cannula insert 300 and prevent movement of the outer cannula 200 relative to the handle 600 about the longitudinal axis of the outer cannula interior bore 220. The shapes capable of being used in other embodiments include, but are not limited to slots, squares, rectangles or triangles or non-symmetrical profiled shapes. Desirably, the handle recess 640 and stabilizing recess 650 are made from rigid material such as plastic or metal, however, these elements may be made from other rigid materials capable of withstanding the manipulative force of the handle 600 onto the outer cannula insert 300. Other rigid materials that could be used for the handle recess 640 or stabilizing recess 650 include, but are not limited to plastics, composites or resins.

As shown in FIG. 4B, the handle 600 further comprises a handle attachment element 670 that mates with the inner rod attachment member 450. In one embodiment, the handle attachment element 670 comprises a protruding nub that is sized and positioned to fit within the inner rod attachment element 460. Alternative embodiments of the handle attachment element 670 include, but are not limited to other methods of mating with the inner rod attachment member 450 such as clips, pins, screws, nubs or other recesses or protrusions.

The Restraining Member:

Generally, the restraining member 800 restrains the outer cannula 200 within the handle 600.

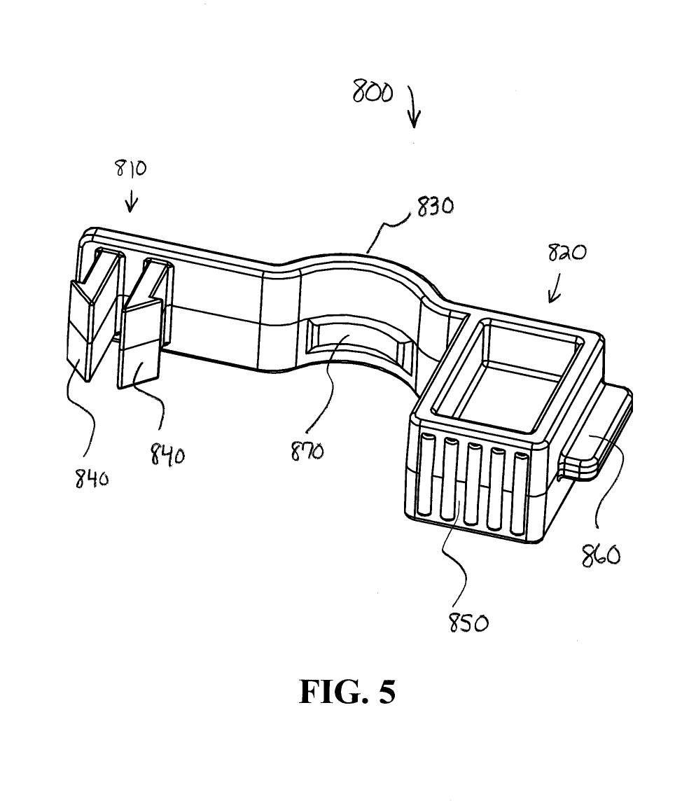

One embodiment of the restraining member 800 shown in FIG. 5 is a flexible element that can be rigidly attached to the handle 600 and shaped to engage the restraining element 330 of the outer cannula insert 300. In this embodiment, the attachment of the restraining member 800 to the handle 600 and its engagement with the restraining element 330 allows the handle 600 to be removably attached to the outer cannula 200.

In this embodiment, the restraining member 800 is a substantially flat and flexible flange with an attachment end 810, a non-attachment end 820 and a semicircular portion 830 having one or more restraining nubs 870. The attachment end 810 is attached to the handle 600 by an attachment means such as, but not limited to, one or more restraining member clips 840 being inserted and secured into the restraining member attachment hole 662. The non-attachment end 820 in this embodiment is not attached to the handle body 610 but positioned in a manner that places the inner diameter of the semicircular portion 830 and the restraining nub 870 over the opening portion 660 of the handle body 610. The semicircular portion 830 connects the two ends of the restraining member 800 and generally has an aperture diameter that when attached to the handle 600 about the opening portion 660, it creates a slightly larger diameter profile than the outer diameter of the outer cannula insert 300. The restraining nub 870 protrudes from the semicircular portion 830 creating a smaller aperture diameter for at least one section of the semicircular portion 830. The resulting aperture diameter for the restraining nub 870 is smaller than the outer diameter of the outer cannula insert 300 but slightly larger than the diameter of the restraining element 330. The shaping of the restraining member 800 allows the restraining nub 870 to fit within the shape of the restraining element 330 of the outer cannula insert 300 therefore allowing the restraining member 800 to restrain the outer cannula insert 300 when it is inserted into the handle 600. The flexibility of the restraining member 800 allows the restraining member's non-attachment end 820, and the semicircular portion 830, to be flexed through a plurality of positions when attached to the handle 600 about the member's attachment end 810. This flexing allows the larger diameter of the outer cannula insert 300 and the outer cannula insert body 380 to pass through the opening portion 660 of the handle body 610 and under the restraining member's semicircular portion 830 and the restraining nub 870. When the restraining element 330 is positioned under the restraining nub 870, the restraining member 800 can be returned to its biased position. Because the aperture of the restraining nub 870 of the restraining member 800 is smaller than the outer diameter of the outer cannula insert 300, the restraining nub 870 fits into the restraining element 330 and restrains the outer cannula insert 300 in the handle 600.

In the embodiment described above, the restraining nub 870 comprises a protruding nub. Alternative embodiments of the restraining nub 870 include, but are not limited to other methods of mating with the restraining element 330 such as clips, pins, screws, buttons or other recesses or protrusions.

The restraining member 800 further comprises a protruding element 850 that enables a user to apply a force to the restraining member 800. The non-attachment end 820 is positioned so that the protruding element 850 is received through the protruding element hole 664 and extends beyond the protruding element hole 664 far enough to allow a user to engage the protruding element 850. The force on the protruding element 850 is used to move the restraining member 800 from its biased position to alternative positions. In one embodiment, the protruding element 850 is a protrusion on the non-attachment end 820 of the restraining member 800. By pushing on the protruding element 850, the restraining member 800 is flexed from its biased position, moving the semicircular portion 830 of the restraining member 800, effectively increasing the diameter profile of the restraining member 800 and allowing the restraining element 330 to move free of the restraining member 800. Other embodiments of the protruding element 850 include, but are not limited to, buttons, pins, toggles, rods, strings or other elements that will allow a force to be transferred to the restraining member 800.

In the embodiments shown in FIGS. 1-5, the positioning of the restraining member 800 in relation to the handle 600 can provide the benefit of the user being able to manipulate the handle 600 and the restraining member 800 with minimal effort. For example, in the embodiments shown, it is possible for a user to hold the handle 600 with the palm of their hand against the upper side 620 while one of the fingers of the user can simultaneously manipulate the restraining member 800 and therefore remove the handle 600 from the outer cannula 200 or outer cannula insert 300. The combination of the flexible nature of the restraining member 800 the use of the protruding element 850 and the positioning of assembly elements enhances the user's ability to operate the assembly 100 with minimal effort. This ease of handle removal also reduces the probability of movement of the outer cannula 200 while it is inserted into the patient and the handle 600 is removed.

One embodiment of the restraining member 800 further comprises a restraining member guide 860, shown in FIG. 5, that is slidably received in a handle guide slot 668 as shown in FIG. 4A. The restraining member guide 860 helps maintain the position of the restraining member 800 relative to the handle 600 when the restraining member 800 is flexed or otherwise stressed.

Other embodiments of the restraining member 800 include any method or shape that allows attachment to the handle 600 and a mating connection to be made with the outer cannula insert restraining element 330. Such methods include, but are not limited to mating notches, protrusions, threads, buttons or other shapes that can mate with a mating restraining element 330 to restrain the outer cannula insert 300 relative to the handle 600. Such other embodiments also include having a rigid restraining member 800 that can be flexibly attached to the handle 600 though such means as springs, hinges, flaps or other flexible attachment means.

Desirably, the retraining member 800 is made from a plastic or metal material. Other materials that could be used for the restraining member 800 include, but are not limited to composites or resins.

Alternative Needle Assembly Embodiment:

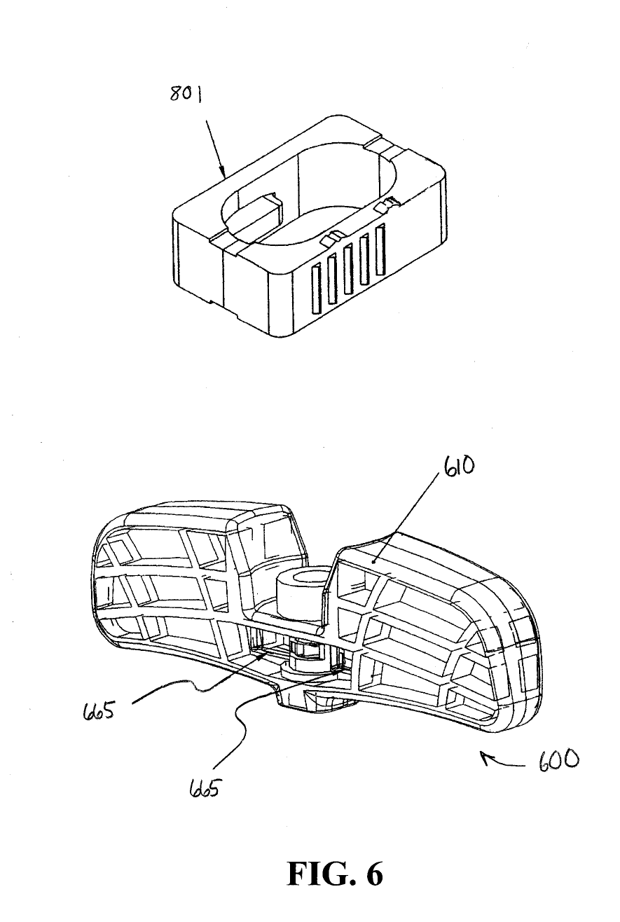

Alternatively, the needle assembly 100 may be designed such as will allow for other methods of restraining the outer cannula insert 300 within the handle 600. One alternative embodiment is shown in FIG. 6 which illustrates an embodiment of the handle 600 and a restraining member 801 that enables a method of restraining the outer cannula insert within the handle through a restraining member capable of sliding through multiple positions relative to other elements of the needle assembly. This embodiment comprises other elements of the needle assembly 100 wherein the restraining member 801 is attached to the handle 600 through one or more handle slide recess 665 and is capable of being moved laterally through a plurality of positions. The restraining member 801 restrains the outer cannula insert 300 within the handle 600 through engagement with restraining elements on the outer cannula insert and one or more restraining nubs on the restraining member 801. The engagement of the restraining member 801 is such that in one position the restraining member 801 allows the outer cannula insert to move in and out of the handle recess while in another position or positions, the restraining member 800 restrains the outer cannula insert within the handle 600. Methods of stabilizing and further restraining the outer cannula 200, the outer cannula insert 300 and the inner rod 400 as described throughout this description can be incorporated into this and similar embodiments.

Other embodiments may incorporate other designs of the restraining member 800 that includes other slidable designs or rotational designs that allow engagement of restraining nubs 870 or similar elements with the restraining elements 330 on the outer cannula insert 300 held within the handle 600.

The Needle Assembly in Operation.

Although it is not necessary for this invention to be used in the operational methods described below, the following description of the operation of one embodiment of the needle assembly 100 is provided to help illustrate one of many embodiments of the invention.

In the embodiment described, the needle assembly 100 comprises an outer cannula 200, an inner rod 400, a handle 600, and a restraining member 800 as shown in FIG. 1. The outer cannula insert 300 in this described embodiment is rigidly attached to the proximal end of the hollow cylindrical element 210 of the outer cannula 200.

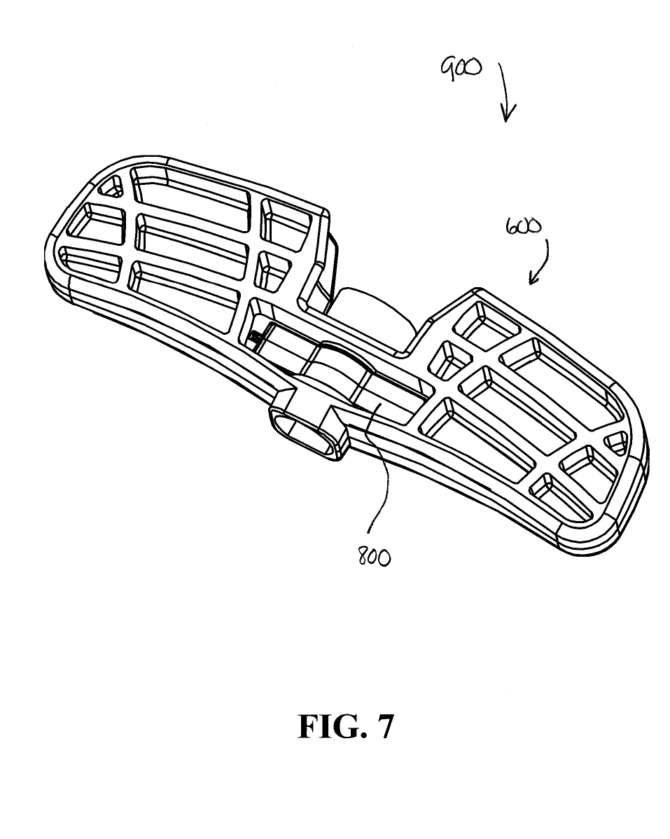

As shown in FIG. 7, the handle 600 and the restraining member 800 can be attached creating a handle assembly 900. The restraining member 800 is attached to the handle 600 utilizing attachment means and elements such as are described elsewhere in this description. Other embodiments of attaching the restraining member 800 to the handle 600 are anticipated including, but not limited to, attachment by the user during the needle assembly's operation or association of the restraining member 800 and the handle 600 with only a temporary attachment of the two elements.

As shown in FIG. 7, the following description of the needle assembly 100 operation describes an embodiment of the handle assembly 900 with the restraining member 800 attached to the handle 600.

The handle assembly 900 is attached to the outer cannula insert 300 by inserting the outer cannula 200 with the outer cannula insert 300 into the handle recess 640 of the handle 600. The insertion is done while aligning the outer cannula insert's distal ring 360 and stabilizing element 340 with the stabilizing recess 650 of the handle 600. In addition, the restraining member 800 is flexed by engaging the protruding element 850 to allow the insertion of outer cannula insert restraining element 330. If the restraining member is not flexed sufficiently, the restraining element 330 will be obstructed by restraining member 800, thereby preventing full insertion of outer cannula insert 300. When inserted into the handle assembly 900, the outer cannula insert 300 passes through the handle recess 640 which is of a sufficient length to accommodate the stabilizing element 340 and the restraining element 330 of the outer cannula insert 300. When inserted, the stabilizing element 340 on the outer cannula insert 300 is aligned with the stabilizing recess 650 whereby the outer cannula insert 300 and the outer cannula 200 are stabilized about the outer cannula interior bore's 220 axis. When the outer cannula insert 300 is fully inserted into the handle recess 640 the outer cannula distal ring 360 and the stabilizing element 340 are retained in and unable to pass beyond the recess bottom portion 642 of the handle recess 640 preventing further insertion of the outer cannula insert 300.

With the outer cannula insert 300 inserted into the handle recess 640, the restraining element 330 of the outer cannula insert 300 is also available for engagement with other elements through the opening portion 660 of the handle 600. The user secures the handle assembly 900 to the outer cannula insert 300 by removing force from the protruding element 850 which allows the restraining member 800 to return to its biased position. Then the restraining member 800 is in its biased position and the restraining element 330 is properly positioned relative to the restraining nub 870, the restraining element 330 receives the restraining nub 870 and restrains the outer cannula insert 300 in place within the handle recess 640. The interoperation of the above elements constrains the movement of the outer cannula insert 300 from movement further into the handle recess 640 by the recess bottom portion 642 and constrains the movement of the outer cannula insert 300 further out of the handle recess 640 by the restraining member 800 engaging the restraining element 330.

The inner rod 400 can be inserted into the proximal end of the outer cannula 200 and attached to the handle attachment element 670 of the handle 600. This is accomplished by engaging the handle attachment element 670 of the handle 600 with the rod attachment element 460 of the inner rod 400. In this embodiment, this is accomplished by aligning the handle attachment element 670 with the rod attachment element 460 and twisting the inner rod. The indented shaping of the rod attachment element 460 retains the handle attachment element 670 in place.

Through this insertion and attachment, the outer cannula insert 300, handle assembly 900 and inner rod 400 are maintained in alignment relative to one another. The engagement of the restraining nub 870 with the restraining element 330 together with the cap receiving end 310 of the outer cannula distal ring 360 abutting the recess bottom portion 642 of the handle recess 640 prevents the outer cannula insert 300 from moving longitudinally within the handle recess 640. The handle attachment element 670 mates with the rod attachment element 460 of the inner rod 400, preventing the inner rod 400 from moving within the outer cannula interior bore 220. And the engagement of the outer cannula insert distal ring 360 with the handle stabilizing recess 650, the outer cannula insert 300, handle assembly 900 and the inner rod 400 are secured about the axis of the cannula's interior bore 220. Therefore, the user of the needle assembly 100 can use a large amount of force, if necessary, to manipulate the entire needle assembly 100 in order to penetrate tissue and/or bone without undesired movement of the outer cannula distal end 230 and inner rod distal end 420 relative to one another.

With the inner rod 400 and the outer cannula 200 restrained in the handle assembly 900, the needle assembly 100 can be easily inserted into the tissue of the patient. This is typically done by manually manipulating the needle assembly 100 in the hands of a user. When the needle assembly 100 is inserted into rigid tissue such as bone, a large amount of force may be needed and striking tools such as a hammer may be used. In one embodiment of this invention, the direct contact of the strike cap 440 and the cylindrical rod 430 of the inner rod 400 provide a method of efficiently transferring this large amount of force directly to the distal end of the inner rod. The direct contact also helps prevent deformation of other elements of the needle assembly 100 due to the force those elements may receive in inserting the assembly.

Once the needle assembly 100 is in place, which is usually determined by the placement of the outer cannula distal end 230, the inner rod 400 is typically removed. The inner rod 400 is removed by disengaging the handle attachment element 670 of the handle 600 from the rod attachment element 460. In this embodiment, this is performed by rotating the inner rod attachment member 450 to disengage the handle attachment element 670 from the inner rod attachment element 460. The inner rod 400 can then be pulled from the interior bore 220 of the outer cannula 200 and the handle recess 640. The result of the removal of the inner rod 400 exposes the interior bore 220 of the outer cannula 200 which can be accessed from the cap receiving end 310 in the outer cannula insert 300. With the outer cannula 200 inserted into the body tissue of the patient, this provides access to the patient's tissue at the outer cannula distal end 230.

In some embodiment of this invention, it is helpful to further attach or insert other tools into the outer cannula 200. This can be accomplished by inserting appropriately shaped elements into the interior bore 220 of the outer cannula 200 or attaching them to the outer cannula insert 300 or the handle 600. These tools may be attached to the outer cannula insert attachment element 350. These tools may also be attached to the handle attachment element 670. These additional tools may provide additional functionality to the needle assembly 100 and may include, but should not be limited to tools such as may be used in obtaining tissue samples.

In one embodiment of this invention, these additional tools include attaching vertebroplasty tools to the assembly 100 to transfer materials into skeletal elements of a patient's body. In these embodiments, elements or tools are attached to the outer cannula insert attachment element 350 through a complementary shaped attachment or other connection means.

In some embodiments of this invention, it is helpful to further remove the handle assembly 900. The handle assembly 900 can be removed from the outer cannula insert 300 by moving the restraining member 800. The protruding element 850 of the restraining member 800 is pushed, moving the non-attachment end 820 of the restraining member 800 and increasing the inside diameter profile of the semicircular portion 830 with the restraining nub 870 such that it is larger than the outer profile of the outer cannula insert 300. This allows the handle assembly 900 to be separated from the outer cannula insert 300 and the outer cannula 200. With the handle assembly 900 removed, this embodiment provides an accessible bore from outside of the patients tissue to the tissue at the outer cannula distal end 230. This accessible bore, represented by the outer cannula 200 and the outer cannula insert 300 has little mass and provides less of an obstruction than a bore with the handle assembly 900 attached.

In some embodiments of this invention, it is helpful to reattach the handle assembly 900. This reattachment may be for additional procedures or it may be to assist in the removal of the needle assembly 100. The handle assembly 900 can be attached to the outer cannula insert 300 and the outer cannula 200 by moving the handle recess 640 over the outer cannula insert 300. The protruding element 850 of the restraining member 800 can be engaged to move the restraining member 800 from its biased position letting the restraining element 330 move into the handle recess 640. When the restraining element 330 is in the proper position, the restraining member 800 can return to its biased position and the restraining nub 870 will restrain the outer cannula insert 300 in the handle recess 640.

In one embodiment, the interoperation of the handle assembly 900 and the outer cannula insert 300 allows the assembly elements to be easily manipulated in the single hand of a user. The protruding element 850 can be accessed by one of the user's fingers while the users hand is still securing the handle assembly 900 allowing the user to remove the handle assembly 900 from the outer cannula insert 300 easily.

In one embodiment, the handle assembly 900 can also be easily reattached to the outer cannula insert 300 with proper shaping of the restraining member semicircular portion 830, the restraining nub 870 and the outer cannula insert 300. These elements may be shaped such that the insertion of the outer cannula insert 300 into the handle recess 640 allows the restraining member's semicircular portion 830 to receive the outer cannula insert 300 and allows the restraining nub 870 to engage the outer diameter of the outer cannula insert 300 and flex the restraining member 800. The flexing can be automatically forced by the force that is being applied to the outer cannula insert 300 as it is being inserted into the handle assembly 900. This flexing will allow insertion of the outer cannula insert 300 until the restraining element 330 is engaged with the restraining nub 870 and the restraining member 800 is returned to its biased position. This simplicity of operation in removing and reattaching the handle assembly 900 is very desirable to the user.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact embodiment and operation shown and described, and accordingly, all suitable modifications and equivalents may be resorted to, falling within the scope of the invention. Although this invention has been described in the above forms with a certain degree of particularity, it is understood that the present disclosure has been made only by way of example and numerous changes in the details of construction and combination and arrangement of parts may be resorted to without departing from the spirit and scope of the invention.

What is claimed is:

1. A needle assembly comprising:
an outer cannula having a restraining element;
a handle;
a restraining member operably attached to the handle; and
the restraining member having a restraining nub whereby the restraining nub engages with the restraining element and the handle can be removably attached to the outer cannula.

2. The needle assembly of claim 1 wherein the restraining member further comprises a flexible element having a protruding element whereby a force applied to the protruding element disengages the restraining nub from the restraining element and the handle can be removably attached to the outer cannula.

3. The needle assembly of claim 2 wherein the handle can be held by at least one appendage of a hand while the force is applied to the protruding element by at least one appendage of the hand.

4. The needle assembly of claim 2 wherein the outer diameter of the outer cannula insert is rounded whereby it can be manufactured by a turning means.

5. The needle assembly of claim 4 wherein the outer cannula insert is manufactured from metal.

6. The needle assembly of claim 2 wherein the outer cannula further comprises a distal end and a threaded proximal end.

7. The needle assembly of claim 2 wherein the outer cannula further comprises a distal end and a proximal end shaped as a luer fitting.

8. The needle assembly of claim 2 further comprising:
a substantially hollow cross-section between a distal end and a proximal end of the outer cannula; and
an inner rod capable of being inserted into and retracted from the hollow cross-section of the outer cannula

9. The needle assembly of claim 8 wherein the inner rod further comprises;
an inner rod proximal end and an inner rod distal end and a rigid rod between the ends; and
a strike cap integrally connected to the rigid rod whereby a force applied to the strike cap is directly transferred to the rigid rod.

10. The needle assembly of claim 9 further comprising:
a rod attachment member connected to the inner rod proximal end;
the rod attachment member having at least one inner rod attachment element; and
the handle having at least one handle attachment element whereby the inner rod attachment element cooperates with the handle attachment element to removably attached the inner rod to the handle.

11. The needle assembly of claim 10 wherein the rod attachment member is non-circular shaped whereby it provides a gripping surface for removable attachment of the inner rod to the handle.

12. A needle assembly comprising:
An inner rod having an inner rod proximal end and an inner rod distal end;
a rigid rod between the inner rod proximal end and inner rod distal end; and
a strike cap integrally connected to the rigid rod whereby a force applied to the strike cap is directly transferred to the rigid rod.

13. The needle assembly of claim 12 further comprising:
an outer cannula having a proximal end, a distal end and a substantially hollow cross-section between the proximal and distal ends;
the substantially hollow cross-section being capable of removably receiving the rigid rod;
a handle;
a rod attachment member connected to the inner rod proximal end;
the rod attachment member having at least one inner rod attachment element; and
the handle having at least one handle attachment element whereby the inner rod attachment element cooperates with the handle attachment element to removably attached the inner rod to the handle.

14. The needle assembly of claim 13 wherein the rod attachment member is non-circular shaped whereby it provides a gripping surface for removable attachment of the inner rod to the handle.

15. The needle assembly of claim 13 further comprising:
the outer cannula having a restraining element;
a restraining member operably attached to the handle; and
the restraining member having a restraining nub whereby the restraining nub engages with the restraining element and the handle can be removably attached to the outer cannula.

16. The needle assembly of claim 13 wherein the outer cannula proximal end is threaded.

17. The needle assembly of claim 13 wherein the outer cannula further comprises a distal end and a proximal end shaped as a luer fitting.

18. The needle assembly of claim 15 wherein the outer diameter of the outer cannula insert is rounded whereby it can be manufactured by a turning means.

19. The needle assembly of claim 18 wherein the outer cannula is manufactured from metal.

20. The needle assembly of claim 15 wherein the restraining member further comprises a flexible element with an attachment end connected to the handle and a non-attached end having a protruding element whereby a force applied to the protruding element disengages the restraining nub from the restraining element and the handle can be removably attached to the outer cannula.

21. The needle assembly of claim 20 wherein the handle can be held by at least one appendage of a hand while the force is applied to the protruding element by at least one appendage of the hand.

22. A needle assembly comprising:
An outer cannula having a restraining element, a substantially hollow cross-section between a distal end and a proximal end of the outer cannula;
an inner rod having an inner rod proximal end, an inner rod distal end, a rigid rod between the inner rod proximal and distal end capable of being inserted into and retracted from the hollow cross-section of the outer cannula;
a strike cap integrally connected to the rigid rod whereby a force applied to the strike cap is directly transferred to the rigid rod;
a rod attachment member connected to the inner rod proximal end;
the rod attachment member having at least one inner rod attachment element;
a handle having at least one handle attachment element whereby the inner rod attachment element cooperates with the handle attachment element to removably attached the inner rod to the handle;
a restraining member operably attached to the handle;
the restraining member having a restraining nub and a protruding element whereby a force applied to the protruding element disengages the restraining nub from the restraining element and the handle can be removed and attached to the outer cannula;
the handle can be held by at least one appendage of a hand while the force is applied to the protruding element by at least one appendage of the hand.

23. The needle assembly of claim 22 wherein the outer diameter of the outer cannula insert is rounded whereby it can be manufactured from metal by a turning means.

24. The needle assembly of claim 22 wherein the outer cannula proximal end is threaded.

25. The needle assembly of claim 22 wherein the outer cannula further comprises a distal end and a proximal end shaped as a luer fitting.

26. The needle assembly of claim 22 wherein the rod attachment member is non-circular shaped whereby it provides a gripping surface for removable attachment of the inner rod to the handle.
